(19) United States
(12) Patent Application Publication
(10) Pub. No.: US 2024/0029136 A1
(43) Pub. Date: Jan. 25, 2024

(54) METHOD, DEVICE, AND COMPUTER PROGRAM FOR RECOMMENDING PRODUCT ON BASIS OF STATE OF ANIMAL

(71) Applicant: DD CARES CO., LTD., Seongnam-si, Gyeonggi-do (KR)

(72) Inventor: Sang Hyun KIM, Seongnam-si (KR)

(73) Assignee: DD CARES CO., LTD., Seongnam-si, Gyeonggi-do (KR)

(21) Appl. No.: 18/026,879

(22) PCT Filed: Sep. 23, 2020
(86) PCT No.: PCT/KR2020/012823

§ 371 Date: Mar. 17, 2023

(51) Int. Cl. G06Q 30/0601; G16H 10/60

(52) U.S. Cl. CPC G06Q 30/0631; G16H 10/60

(57) ABSTRACT

Provided is a method of recommending a product based on the condition of an animal, the method including receiving basic information regarding the animal and disease information regarding the animal, estimating a current condition of the animal based on the basic information and the disease information, and recommending the products corresponding to the current condition.

### TECHNICAL FIELD

The present disclosure relates to a method, an apparatus, and a computer program for recommending a product based on the condition of an animal.

### BACKGROUND ART

The population that recognizes and manages animals, especially dogs and cats, as companion animals is continuously increasing. Recently, a large number of animals suffer from obesity, arthritis, separation anxiety, skin diseases, and allergies, but, due to limitations in communication with animals, it is difficult to detect health problems at the time of initial symptoms, and thus health of animals is deteriorated and treatment costs increase.

There are various ways to manage animals. One of the common method of managing animals is to provide simple management functions like simply providing a video or putting food in a container for an animal left alone while an owner is not at home.

Therefore, beyond common animal management methods, a technology that can help in managing the health of animals and preventing diseases by monitoring the health conditions of animals in real time is demanded.

### DESCRIPTION OF EMBODIMENTS

### Technical Problem

The present disclosure provides a method, an apparatus, and a computer program for recommending a product based on the condition of an animal. The technical goals to be achieved by the present embodiment are not limited to the technical goals as described above, and other technical problems may be inferred from the following embodiments.

### Solution to Problem

According to an embodiment of the disclosure, a method of recommending a product based on the condition of an animal, the method includes receiving basic information regarding the animal and disease information regarding the animal; estimating a current condition of the animal based on the basic information and the disease information; and recommending the products corresponding to the current condition.

In the estimating, the current condition is estimated by combining a plurality of first elements included in the basic information and a plurality of second elements included in the disease information.

In the estimating, the current condition is estimated by applying a certain weight to each of the first elements and the second elements.

The basic information includes at least one of the type of the animal, the breed of the animal, the age of the animal, the sex of the animal, the real-time condition of the animal, and the neuter status of the animal.

The disease information includes at least one of a disease which the animal currently suffers from, a disease which the animal has suffered from in the past, and an allergy of the animal.

The method further includes providing a reward to a user based on whether the user has purchased a recommended product.

According to another embodiment of the disclosure, there is provided a computer-readable recording medium having recorded thereon a program for executing the method on a computer.

According to another embodiment of the disclosure, an apparatus for recommending a product based on the condition of an animal, the apparatus includes a communication interface for receiving basic information regarding the animal and disease information regarding the animal; a memory; and a processor configured to estimate a current condition of the animal based on the basic information and the disease information and recommend products corresponding to the current condition.

The processor estimates the current condition by combining a plurality of first elements included in the basic information and a plurality of second elements included in the disease information.

The processor estimates the current condition by applying a certain weight to each of the first elements and the second elements.

The basic information includes at least one of the type of the animal, the breed of the animal, the age of the animal, the sex of the animal, the real-time condition of the animal, and the neuter status of the animal.

The communication interface receives a real-time condition of the animal from a wearable device.

The disease information includes at least one of a disease which the animal currently suffers from, a disease which the animal has suffered from in the past, and an allergy of the animal.

The processor provides a reward to a user based on whether the user has purchased a recommended product.

### Advantageous Effects of Disclosure

According to the present disclosure, products (e.g., feeds, nutritional supplements, etc.) corresponding to the current condition of an animal may be recommended to a user. Therefore, the health of an animal may be improved through products reflecting the current condition of the animal, a disease which the animal currently suffers from, and matters that need to be improved.

In an embodiment, according to the present disclosure, a reward may be provided to a user who purchases recommended products. Therefore, not only an incentive to purchase recommended products may be provided to a user, but also the user may utilize the provided reward to purchase other products.

### BRIEF DESCRIPTION OF DRAWINGS

FIG. 1 is a diagram showing a system including a user terminal and a wearable device according to an embodiment.

FIG. 2 is a diagram showing an example of transmitting real-time condition information regarding an animal from a wearable device to a user terminal according to an embodiment.

FIG. 3 is a diagram showing an example of a product recommending apparatus according to an embodiment.

FIG. 4 is a flowchart showing an example of a method of recommending products based on the condition of an animal, according to an embodiment.

FIG. 5 is a diagram for describing an example in which basic information and disease information regarding an animal are stored in a memory according to an embodiment.

FIG. 6 is a diagram for describing an example in which the real-time condition of an animal is output according to an embodiment.

FIG. 7 is a diagram for describing an example in which a processor estimates the current condition of an animal, according to an embodiment.

FIG. 8 is a diagram for describing an example in which a processor recommends products according to an embodiment.

FIG. 9 is a diagram for describing an example in which a system recommends products according to an embodiment.

### BEST MODE

According to an embodiment of the disclosure, a method of recommending a product based on the condition of an animal, the method includes receiving basic information regarding the animal and disease information regarding the animal; estimating a current condition of the animal based on the basic information and the disease information; and recommending the products corresponding to the current condition.

### Mode of Disclosure

Hereinafter, exemplary embodiments of the disclosure will be described in detail with reference to the accompanying drawings. The disclosure may, however, be embodied in many different forms and should not be construed as limited to the embodiments set forth herein. In order to clearly illustrate the disclosure, parts not related to the description are omitted, and like parts are denoted by like reference numerals throughout the specification.

Throughout the specification, it will be understood that when a portion is referred to as being “connected to” another portion, it can be “directly connected to” the other portion or “electrically connected to” the other portion via another element. In addition, unless explicitly described to the contrary, the word “comprise” and variations such as “comprises” or “comprising” will be understood to imply the inclusion of stated elements but not the exclusion of any other elements.

Hereinafter, the disclosure will be described in detail with reference to accompanying drawings.

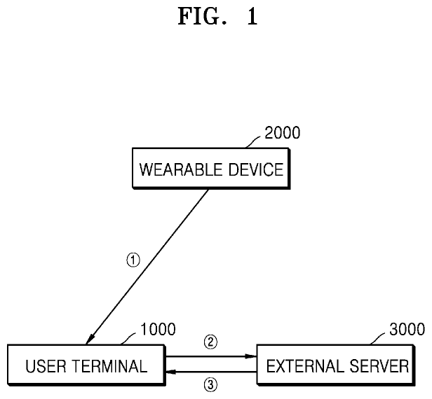

FIG. 1 is a diagram showing a system including a user terminal and a wearable device according to an embodiment.

A system according to an embodiment may include a user terminal 1000 and a wearable device 2000. In an embodiment, the system may further include an external server 3000.

The user terminal 1000, the wearable device 2000, and the external server 3000 may perform communication with one another by using a network. For example, the network is a comprehensive data communication network, which includes a local area network (LAN), a wide area network (WAN), a value added network (VAN), a mobile radio communication network, a satellite communication network, and mutual combinations thereof, enables smooth communication between the network constituent entities shown in FIG. 1, and may include a wired internet, a wireless internet, and a mobile wireless communication network. In an embodiment, wireless communication may include, but is not limited to, for example, wireless LAN (Wi-Fi), Bluetooth, Bluetooth low energy, Zigbee, Wi-Fi Direct (WFD), ultra wideband (UWB), infrared data association (IrDA), and Near Field Communication (NFC).

The user terminal 1000 may include, but is not limited to, any one of a smartphone, a tablet PC, a PC, a smart TV, a mobile phone, a personal digital assistant (PDA), a laptop computer, a media player, a micro server, a global positioning system (GPS) device, an e-book reader, a digital broadcasting terminal, a navigation device, a kiosk, an MP3 player, a digital camera, a home appliance, a device having a camera, and other mobile or non-mobile computing devices.

The wearable device 2000 may be worn on a body part of an animal to collect real-time condition information of the animal. The wearable device 2000 may include a plurality of sensors. For example, the wearable device 2000 may include at least one of an electromyography (EMG) sensor, an electrodermal activity sensor, a skin temperature measuring device, a blood volume pulse measuring device, an electrocardiogram (ECG) sensor, a respiration sensor, a blood pressure measuring device, and a heart rate measuring device. In an embodiment, the wearable device 2000 may include a 3-axis accelerometer, a 6-axis gyroscope & accelerometer, and a 9-axis gyroscope & accelerometer & geomagnetic sensor.

The external server 3000 may communicate with the user terminal 1000 and the wearable device 2000 through a network.

According to an embodiment, referring to a first arrow, the wearable device 2000 may transmit real-time condition information regarding an animal to the user terminal 1000. The wearable device 2000 may transmit real-time condition information regarding an animal to the user terminal 1000 through a communication method like Wi-Fi, Bluetooth, and Bluetooth low energy.

Referring to a second arrow, the user terminal 1000 may transmit real-time condition information to the external server 3000 and request recommendation of a product that matches the condition of the animal. The external server 3000 may analyze basic information and disease information regarding the animal to estimate the current state of the animal and may recommend products corresponding to the current condition. Here, the products may be feeds or nutritional supplements for the animal.

Referring to the third arrow, the external server 3000 transmits information regarding the current state of the animal and information regarding recommended products to the user terminal 1000, and the user terminal 1000 may inform a user of the current condition of the animal and recommended products corresponding to the current condition. The user terminal 1000 and the external server 3000 may exchange data through wired Internet, wireless Internet, or a mobile wireless communication network.

According to another embodiment, referring to the first arrow, real-time condition information regarding an animal obtained by the wearable device 2000 may be transmitted to the user terminal 1000. At this time, instead of transmitting the real-time condition information to the external server 3000, the user terminal 1000 may estimate the current condition of the animal on its own and provide recommended products corresponding to the current condition to a user.

According to another embodiment, although not shown in FIG. 1, the wearable device 2000 may transmit real-time condition information directly to the external server 3000 and request recommendation of products corresponding to the condition of the animal, without going through the user terminal 1000. In this case, the external server 3000 may transmit the current condition of the animal and recommended products corresponding to the current condition to the user terminal 1000, and the user terminal 1000 may provide the current condition of the animal and the recommended products corresponding to the current condition to a user. The user terminal 1000, the wearable device 2000, and the external server 3000 may exchange data through wired Internet, wireless Internet, or a mobile wireless communication network.

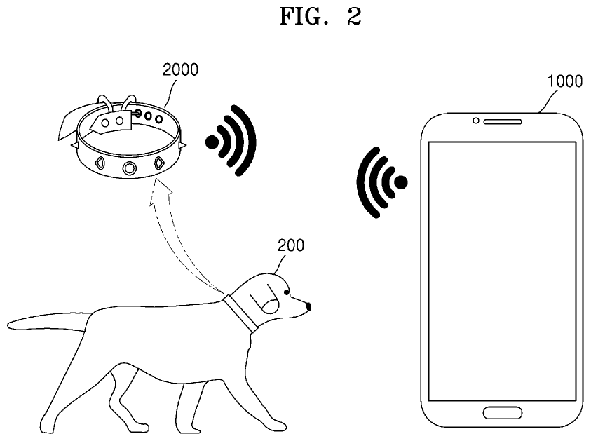

FIG. 2 is a diagram showing an example of transmitting real-time condition information regarding an animal from a wearable device to a user terminal according to an embodiment.

Referring to FIG. 2, a wearable device 2000 may be worn on a body part of an animal 200 to collect real-time condition information regarding the animal 200.

In one embodiment, the real-time condition information may be information indicating whether the animal 200 is currently in an active state or a sleeping state. More specifically, the real-time condition information may be information indicating whether the animal 200 is in a play state, an active state, a resting state, or a sleeping state. However, the type of real-time condition information is not limited thereto.

The wearable device 2000 may include at least one sensor, and the wearable device 2000 may collect real-time condition information regarding the animal 200 based on a sensed value of the at least one sensor.

For example, the wearable device 2000 may include at least one of an EMG sensor, an electrodermal activity sensor, a skin temperature measuring device, a blood volume pulse measuring sensor, an ECG sensor, a respiration sensor, a blood pressure measuring device, a heart rate measuring device, a 3-axis accelerometer, a 6-axis gyroscope/accelerometer, and a 9-axis gyroscope/accelerometer/geomagnetic sensor. The EMG sensor refers to a sensor that detects an action potential of a muscle. The electrodermal activity sensor refers to a sensor that measures skin conductivity. The skin temperature measuring device may include a sensor that detects the temperature of the skin surface. The blood volume pulse measuring sensor refers to a device that measures the amount of blood flowing in a blood vessel. The ECG sensor refers to a sensor that senses an electrical potential related to heartbeat on a body surface. The respiration sensor refers to a sensor that measures how much and quickly one breathes. The heart rate measuring device refers to a device that measures the number of times the heart beats per unit time. The 3-axis accelerometer, the 6-axis gyroscope/accelerometer, and the 9-axis gyroscope/accelerometer/geomagnetic sensor refers to sensors that measure dynamic forces like acceleration and vibration impact.

The user terminal 1000 may receive real-time condition information regarding the animal 200 from the wearable device 2000.

The user terminal 1000 may determine the current condition of the animal 200 based on real-time condition information. For example, the current state of the animal 200 may include whether the animal 200 has a disease (e.g., arthritis, skin disease, allergy, separation anxiety, etc.) and/or the constitution of the animal 200 (e.g., an allergy to a specific ingredient, etc.), but is not limited thereto.

In an embodiment, the user terminal 1000 may provide a solution according to the current condition of the animal 200. The solution may be information regarding feed or nutritional supplements helpful for the health of the animal 200.

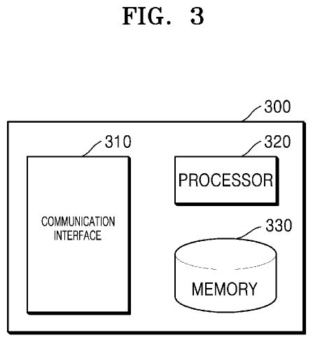

FIG. 3 is a diagram showing an example of a product recommending apparatus according to an embodiment.

Referring to FIG. 3, a product recommending apparatus 300 includes a communication interface 310, a processor 320, and a memory 330. Only components related to the embodiment are shown in the product recommending apparatus 300 of FIG. 3. Therefore, one of ordinary skill in the art will understand that other general-purpose components may be further included in the product recommending apparatus 300 in addition to the components shown in FIG. 3.

In an embodiment, the product recommending apparatus 300 may be the user terminal 1000 or the external server 3000 described above with reference to FIG. 1. Therefore, it would be obvious that, even when omitted below, descriptions given above with respect to the user terminal 1000 or the external server 3000 shown in FIG. 1 are also applicable to the product recommending apparatus 300 of FIG. 3.

The communication interface 310 may include one or more components that perform wired or wireless communication with an external device. For example, the communication interface 310 may include at least one of a short-range communication unit (not shown), a mobile communication unit (not shown), and a broadcast reception unit (not shown).

For example, the communication interface 310 may receive basic information and disease information regarding the animal 200 from an external device. When the product recommending apparatus 300 is the user terminal 1000, the external device may be the external server 3000 or the wearable device 2000. Alternatively, when the product recommending apparatus 300 is the external server 3000, the external device may be the user terminal 1000 or the wearable device 2000.

Meanwhile, in the above example, it has been described that the product recommending apparatus 300 receives basic information and disease information of the animal 200 from an external device. However, the disclosure is not limited thereto. In other words, the basic information and disease information regarding the animal 200 may be obtained or produced by the product recommending apparatus 300 itself.

The memory 330 is hardware for storing various data processed in the product recommending apparatus 300 and may store programs for processing and controlling the processor 320.

For example, the memory 330 may store basic information regarding the animal 200, disease information regarding the animal 200, information regarding the current condition of the animal 200, and information regarding recommended products.

The memory 330 may include a random access memory (RAM), such as a dynamic random access memory (DRAM) and a static random access memory (SRAM), a read-only memory (ROM), an electrically erasable programmable read-only memory (EEPROM), a CD-ROM, a Blu-ray or another optical disc storage, a hard disk drive (HDD), a solid state drive (SSD), or a flash memory.

The processor 320 controls the overall operation of the product recommending apparatus 300. For example, the processor 320 may generally control an input unit (not shown), a display (not shown), the communication interface 310, and the memory 330 by executing programs stored in the memory 330. The processor 320 may control the operation of the product recommending apparatus 300 by executing programs stored in the memory 330.

For example, the processor 320 may estimate the current condition of the animal 200 based on basic information and disease information regarding the animal 200. Then, the processor 320 may recommend products corresponding to the current condition of the animal 200. In an embodiment, the processor 320 may provide a reward to a user based on whether the user has purchased a recommended product.

For example, the processor 320 may be implemented by using at least one of application specific integrated circuits (ASICs), digital signal processors (DSPs), digital signal processing devices (DSPDs), programmable logic devices (PLDs), field programmable gate arrays (FPGAs), controllers, micro-controllers, microprocessors, and other electrical units for performing functions.

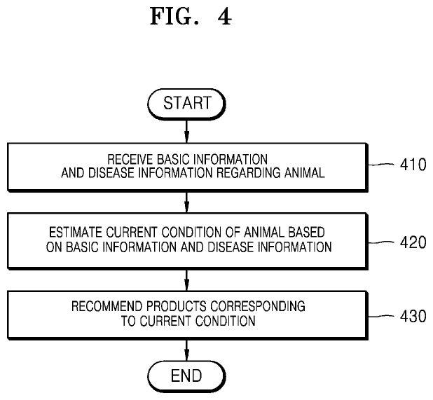

Hereinafter, with reference to FIGS. 4 to 9, a method by which the processor 320 recommends products based on the condition of an animal will be described in detail.

FIG. 4 is a flowchart showing an example of a method of recommending products based on the condition of an animal, according to an embodiment.

Referring to FIG. 4, the method of recommending products based on the condition of the animal 200 includes operations performed in time series by the user terminal 1000, the wearable device 2000, or the external server 3000 shown in FIG. 3. Therefore, even when descriptions thereof are omitted below, descriptions given above with regard to the user terminal 1000, the wearable device 2000, or the external server 3000 shown in FIGS. 1 and 2 are also applicable to the method of recommending products based on the condition of the animal 200 of FIG. 4.

In operation 410, the communication interface 310 receives basic information regarding the animal 200 and disease information regarding the animal 200.

The basic information regarding the animal 200 includes at least one of the type of the animal 200, the breed of the animal 200, the age of the animal 200, the sex of the animal 200, the real-time condition of the animal 200, and the neuter status of the animal 200. For example, assuming that the animal 200 is a dog, the type of the animal 200 indicates a dog, and the breed of the animal 200 indicates a Doberman, Poodle, Bulldog, Alaskan Malamute, etc.

Meanwhile, information regarding the real-time condition of the animal 200 may be collected by the wearable device 2000 and received through the communication interface 310. For example, the real-time condition information may be information indicating whether the animal 200 is in a play state, an active state, a resting state, or a sleeping state. However, the type of real-time condition information is not limited to the above-stated examples. The example of collecting information regarding the real-time condition of the animal 200 by the wearable device 2000 is as described above with reference to FIG. 2.

The disease information regarding the animal 200 includes at least one of a disease the animal 200 currently suffers from, a disease the animal 200 has suffered from in the past, and an allergy of the animal 200. Here, the type of disease is not limited in any way, and the cause of an allergy is not limited to specific examples.

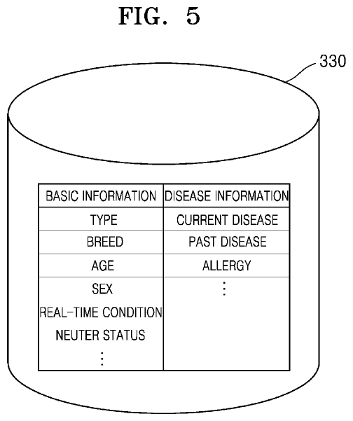

Meanwhile, the basic information and the disease information regarding the animal 200 may be stored in the memory 330. Hereinafter, an example in which basic information and disease information regarding the animal 200 are stored in the memory 330 will be described with reference to FIG. 5.

FIG. 5 is a diagram for describing an example in which basic information and disease information regarding an animal are stored in a memory according to an embodiment.

FIG. 5 shows an example in which basic information and disease information regarding the animal 200 are stored in the memory 330. Basic information and disease information may include various types of information. Therefore, the memory 330 may distinguish various information included in the basic information and various information included in the disease information from each other and store them. In an embodiment, when a user is raising a plurality of animals 200, basic information and disease information regarding the respective animals 200 may be stored separately from one another in the memory 330.

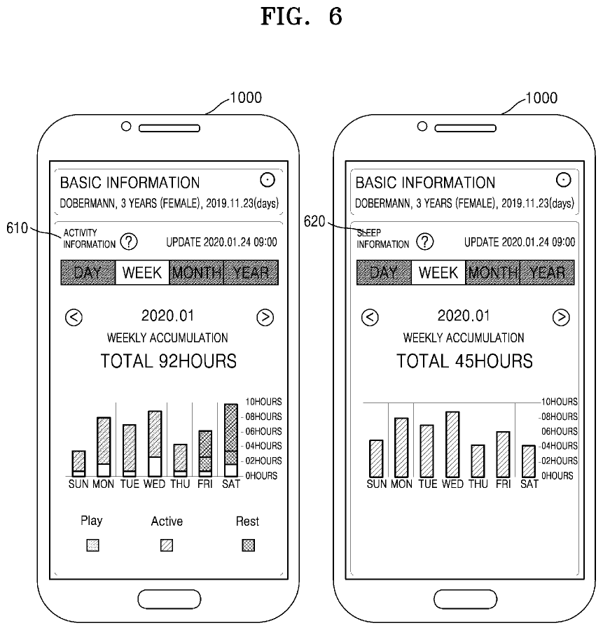

Meanwhile, the processor 320 may output information regarding the real-time condition of the animal 200 through a display, and the user may check the real-time condition of the animal 200 based on output information. Hereinafter, an example in which the processor 320 outputs information regarding the real-time condition of the animal 200 will be described with reference to FIG. 6.

FIG. 6 is a diagram for describing an example in which the real-time condition of an animal is output according to an embodiment.

The communication interface 310 may receive the real-time condition information regarding the animal 200 from the wearable device 2000, and the processor 320 may output the real-time condition information regarding the animal 200 through a display.

For example, referring to FIG. 3, the user terminal 1000 may receive real-time condition information regarding an animal from the wearable device 2000, accumulate received real-time condition information on a daily, weekly, monthly, and yearly basis, and provide accumulated information to a user.

According to an embodiment, the user terminal 1000 may aggregate accumulated time for each of a play state, an active state, and a resting state in the real-time condition information and provide aggregated times to a user as activity information 610. In an embodiment, the user terminal 1000 may accumulate time for a sleeping state in the real-time condition information and provide accumulated time to a user as sleep information 620.

Meanwhile, the user terminal 1000 may not only provide real-time condition information regarding the animal 200 to a user, but also determine the type of health abnormality of the animal 200 based on the real-time condition information.

For example, when a result of analyzing activity information 610 in the real-time condition information indicates that the time corresponding to a play state of the animal 200 is equal to or greater than a critical time, the user terminal 1000 may determine the type of health abnormality of the animal 200 as separation anxiety.

In another example, when a result of analyzing the activity information 610 indicates that the time corresponding to a resting state of the animal 200 is equal to or greater than a critical time, the user terminal 1000 may determine the type of health abnormality of the animal 200 as arthritis.

However, the user terminal 1000 may determine the type of health abnormality of the animal 200 in consideration of various sensed values received from the wearable device 2000 as well as times corresponding to the play state, the active state, the resting state, and the sleeping state of the animal 200. For example, the user terminal 1000 may determine the type of health abnormality of the animal 200 in consideration of the sensed values received from an EMG sensor, an electrodermal activity sensor, a skin temperature measuring device, a blood pressure measuring device, an accelerometer, etc. of the wearable device 2000.

Referring back to FIG. 4, in operation 420, the processor 320 estimates the current condition of the animal 200 based on the basic information and the disease information.

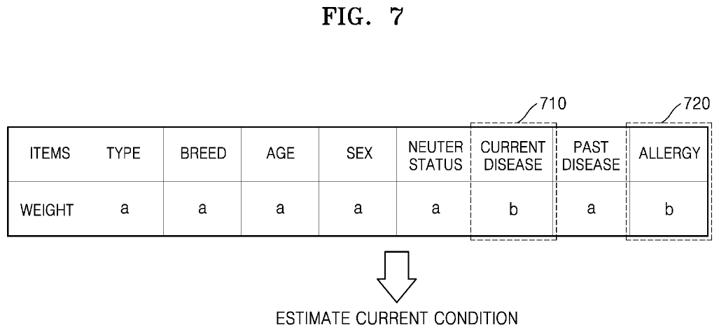

The processor 320 may estimate the current condition of the animal 200 by considering all of the basic information and the disease information stored in the memory 330 or selecting some of them. For example, the processor 320 may estimate the current condition of the animal 200 by combining a plurality of first elements (information) included in the basic information and a plurality of second elements (information) included in the disease information. At this time, the processor 320 may estimate the current condition of the animal 200 by applying a certain weight to each of the first elements and the second elements. Hereinafter, an example in which the processor 320 estimates the current condition of the animal 200 will be described with reference to FIG. 7.

FIG. 7 is a diagram for describing an example in which a processor estimates the current condition of an animal, according to an embodiment.

FIG. 7 shows various types of basic information and disease information regarding the animal 200. For example, various types of basic information and disease information may include the type, the breed, the age, the sex, the neuter status, a current disease, a past disease, an allergy, etc. of the animal 200.

The processor 320 may estimate the current condition of the animal 200 by combining various types of information regarding the animal 200. At this time, the processor 320 may estimate the current condition of the animal 200 by applying a certain weight to each of the various types of information.

For example, the processor 320 may set weights for a current disease 710 and an allergy 720 of the animal 200 differently from weights for other information. In general, the current disease 710 and the allergy 720 are identified as very important factors in estimating the current condition of the animal 200. Therefore, the processor 320 may assign higher weights to the current disease 710 and allergy 720 than other information. In this case, in estimating the current condition of the animal 200, the current disease 710 and allergy 720 may have greater influences than other information.

In an embodiment, although FIG. 7 shows that the current disease 710 and allergy 720 have the same weight, the disclosure is not limited thereto. In other words, when the current disease 710 is more important information for estimating the current condition of the animal 200, the processor 320 may assign a higher weight to the current disease 710 than to the allergy 720.

Referring back to FIG. 4, in operation 430, the processor 320 recommends products corresponding to the current condition of an animal.

Here, the products may be feeds or nutritional supplements for the animal 200. As described above with reference to operation 420, the processor 320 estimates the current condition of the animal 200. Therefore, products recommended by the processor 320 may be products helpful for the health of the animal 200. Therefore, a user may not only check the current condition of his or her companion animal, but also check products that may solve health problems of the companion animal.

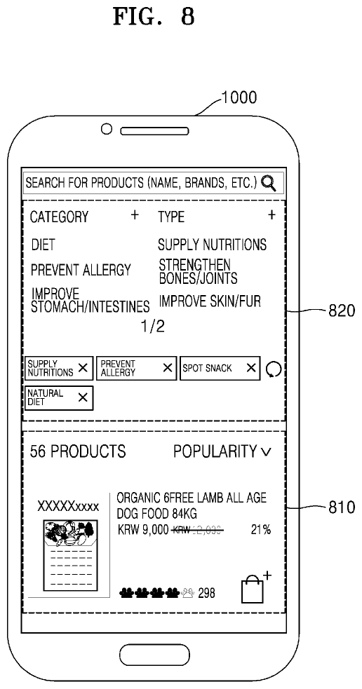

Hereinafter, an example in which the processor 320 recommends products corresponding to the current condition of an animal will be described with reference to FIG. 8.

FIG. 8 is a diagram for describing an example in which a processor recommends products according to an embodiment.

The user terminal 1000 may provide information 810 regarding products helpful for the health of the animal 200 as a solution based on the current condition of the animal 200. For example, assuming that products are feeds for the animal 200, the user terminal 1000 may provide necessary nutrient information, feed product information, etc. as the information 810.

In detail, when the animal 200 currently has obesity, the user terminal 1000 may provide the information 810 regarding nutrient information for diet or feed products for diet. Alternatively, when the animal 200 currently suffers from arthritis, the user terminal 1000 may provide the information 810 regarding nutrients for strengthening bones/joints or feed products for improving arthritis.

For example, when a user purchases a feed product provided as the information 810, the user terminal 1000 may provide a reward. At this time, the user may use the reward to purchase other products.

Meanwhile, a user may select an additional condition 820. The user terminal 1000 may receive an input for selecting the additional condition 820 by a user. When the additional condition 820 is determined, the user terminal 1000 may filter and provide only feed products that satisfy the additional condition 820 from among feed products provided as the information 810 to the user.

For example, when the animal 200 currently suffers from arthritis, the user terminal 1000 may provide feed products for improving arthritis by strengthening bones/joints as the information 810. At this time, when ‘natural diet’ is determined as the additional condition 820, the user terminal 1000 may filter and provide only with feed products satisfying the condition ‘natural diet’ from among the feed products for improving arthritis to the user.

The user may use rewards points to purchase feed products offered by a solution. In an embodiment, when a user purchases a product offered by a solution, the user terminal 1000 may provide a reward to the user.

Although not shown in FIG. 5, the user terminal 1000 may provide animal insurance information as a solution based on the current condition of an animal. Animal insurance information provided by the user terminal 1000 may include insurance plan information, etc. For example, when the animal 200 currently suffers from arthritis, the user terminal 1000 may provide a user with an insurance plan including arthritis coverage. A user may use rewards points to purchase an insurance plan offered by a solution. In an embodiment, when a user purchases an insurance plan offered as a solution, the user terminal 1000 may provide a reward to the user.

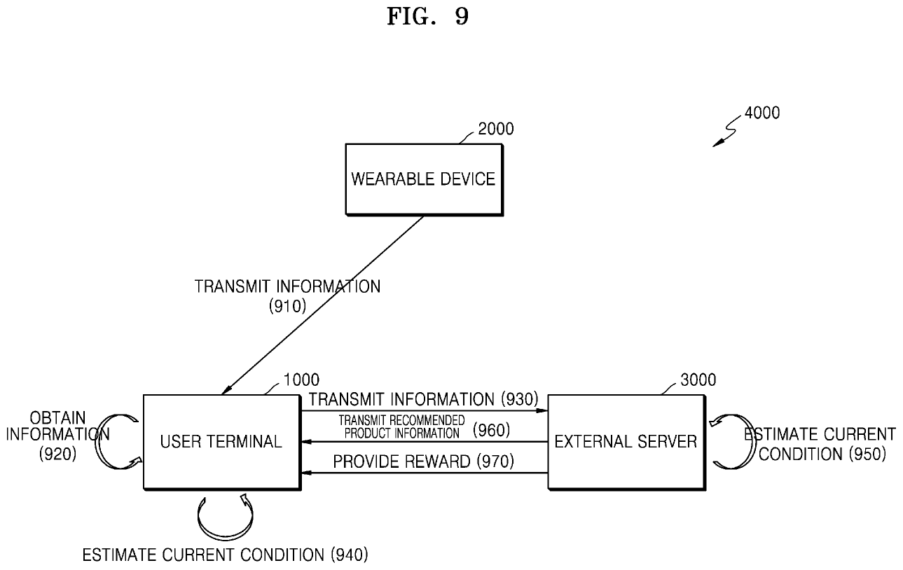

FIG. 9 is a diagram for describing an example in which a system recommends products according to an embodiment.

Referring to FIG. 9, a system 4000 includes the user terminal 1000, the wearable device 2000, and the external server 3000. Regarding the system 4000 of FIG. 9, only components related to an embodiment are illustrated. Therefore, one of ordinary skill in the art could understand that other general-purpose components may be further included in the system 4000 in addition to the components shown in FIG. 9.

Meanwhile, the product recommending apparatus 300 shown in FIG. 3 may correspond to the user terminal 1000 or the external server 3000 of the system 4000. Therefore, even when descriptions are omitted below, descriptions of the user terminal 1000 or the external server 3000 given above with reference to FIGS. 1 to 8 are also applicable to the system 4000 of FIG. 9.

The wearable device 2000 transmits information regarding the animal 200 to the user terminal 1000 (910). For example, the wearable device 2000 may transmit information regarding the animal 200 to the user terminal 1000 through a wireless or wired communication method. Here, the information regarding the animal 200 may include basic information and disease information regarding the animal 200.

The user terminal 1000 may obtain information regarding the animal 200 (920). In other words, the user terminal 1000 may receive information regarding the animal 200 from the wearable device 2000 or the user terminal 1000 may obtain information on its own through a user input.

The user terminal 1000 transmits information regarding the animal 200 to the external server 3000 (930). For example, the user terminal 1000 may transmit information regarding the animal 200 to the external server 3000 through a wireless or wired communication method.

The user terminal 1000 may estimate the current condition of the animal 200 by using information regarding the animal 200 transmitted from the wearable device 2000 or obtained on its own (940). Alternatively, the external server 3000 may estimate the current condition of the animal 200 by using information regarding the animal 200 transmitted from the user terminal 1000 (950). When the user terminal 1000 has estimated the current condition of the animal 200, the user terminal 1000 may transmit information regarding an estimated current condition to the external server 3000.

The external server 3000 transmits information regarding recommended products to the user terminal 1000 (960). Here, the products means feeds or nutritional supplements for the animal 200.

The user terminal 1000 may display information regarding recommended products transmitted from the external server 3000, and a user may decide whether to purchase the recommended products. When the user purchases a recommended product, the external server 3000 may provide a reward to the user through the user terminal 1000 (970).

Various embodiments of the present disclosure may be implemented as software (e.g., a program) including one or more instructions stored in a machine-readable storage medium. For example, a processor of a device may invoke at least one instruction from among one or more instructions stored in a storage medium and execute it. This enables the device to be operated to perform at least one function according to the at least one instruction invoked. The one or more instructions may include codes generated by a compiler or codes executable by an interpreter. The machine-readable storage medium may be provided in the form of a non-transitory storage medium. Here, the term ‘non-temporary’ merely means that a storage medium is a tangible device and does not contain signals (e.g., electromagnetic waves), and this term does not discriminate the case where data is stored semi-permanently in a storage medium and the case where data is temporarily stored in a storage medium.

According to an embodiment, the method according to various embodiments of the present disclosure may be included and provided in computer program products. Computer program products may be traded between sellers and buyers as commodities. Computer program products may be distributed in the form of a device-readable storage medium (e.g., compact disc read-only memory (CD-ROM)), on-line (e.g., download or upload) through an application store (e.g., PlayStore™), or directly between two user devices. In the case of online distribution, at least a part of a computer program product may be temporarily stored or temporarily created in a machine-readable storage medium like a memory of a manufacturer's server, an application store server, or a relay server.

In this specification, a “unit” may be a hardware component such as a processor or a circuit, and/or software component executed on the hardware like a processor.

The scope of the disclosure is indicated by the claims which will be described in the following rather than the detailed description of the exemplary embodiments, and it should be understood that the claims and all modifications or modified forms drawn from the concept of the claims are included in the scope of the disclosure.

1. A method of recommending a product based on a condition of an animal, the method comprising:
receiving basic information regarding the animal and disease information regarding the animal;
estimating a current condition of the animal based on the basic information and the disease information; and
recommending the product corresponding to the current condition.

2. The method of claim 1, wherein, in the estimating, the current condition is estimated by combining a plurality of first elements included in the basic information and a plurality of second elements included in the disease information.

3. The method of claim 2, wherein, in the estimating, the current condition is estimated by applying a certain weight to each of the first elements and the second elements.

4. The method of claim 1, wherein the basic information comprises at least one of a type of the animal, a breed of the animal, an age of the animal, a sex of the animal, a real-time condition of the animal, and a neuter status of the animal.

5. The method of claim 1, wherein the disease information comprises at least one of a disease which the animal currently suffers from, a disease which the animal has suffered from in the past, and an allergy of the animal.

6. The method of claim 1, further comprising providing a reward to a user based on whether the user has purchased a recommended product.

7. A computer-readable recording medium having recorded thereon a program for executing the method of claim 1 on a computer.

8. An apparatus for recommending a product based on a condition of an animal, the apparatus comprising:
a communication interface for receiving basic information regarding the animal and disease information regarding the animal;
a memory; and
a processor configured to estimate a current condition of the animal based on the basic information and the disease information and recommend products corresponding to the current condition.

9. The apparatus of claim 8, wherein the processor estimates the current condition by combining a plurality of first elements included in the basic information and a plurality of second elements included in the disease information.

10. The apparatus of claim 9, wherein the processor estimates the current condition by applying a certain weight to each of the first elements and the second elements.

11. The apparatus of claim 8, wherein the basic information comprises at least one of a type of the animal, a breed of the animal, an age of the animal, a sex of the animal, a real-time condition of the animal, and a neuter status of the animal.

12. The apparatus of claim 11, wherein the communication interface receives a real-time condition of the animal from a wearable device.

13. The apparatus of claim 8, wherein the disease information comprises at least one of a disease which the animal currently suffers from, a disease which the animal has suffered from in the past, and an allergy of the animal.

14. The apparatus of claim 8, wherein the processor provides a reward to a user based on whether the user has purchased a recommended product.
